(19) United States
(12) Patent Application Publication
(10) Pub. No.: US 2018/0272011 A1
(43) Pub. Date: Sep. 27, 2018

(54) MRI CONTRAST AGENT AND METHOD FOR PREPARING THE SAME

(71) Applicant: Huazhong University of Science and Technology, Wuhan (CN)

(72) Inventors: Qibing ZHOU, Wuhan (CN); Yushuang WEI, Wuhan (CN); Xiangliang YANG, Wuhan (CN)

(21) Appl. No.: 15/995,111

(22) Filed: May 31, 2018

Related U.S. Application Data
(63) Continuation-in-part of application No. PCT/CN2015/098234, filed on Dec. 22, 2015.

(30) Foreign Application Priority Data
Dec. 1, 2015 (CN) 201510870335.3

(51) Int. Cl. A61K 49/18; A61K 49/12

(52) U.S. Cl. CPC A61K 49/1872; B82Y 15/00; A61K 49/12

(57) ABSTRACT

An MRI contrast agent, including superparamagnetic nanoparticles and hydroxyethyl starch. The weight ratio of the superparamagnetic nanoparticles to the hydroxyethyl starch is between 1:5 and 1:15. The superparamagnetic nanoparticles have a particle size of 100-140 nm, and include the following layers, from the inside out, ferrous ferric oxide particles, citric acid, and poly-R-lysine. The citric acid accounts for 6-13 wt. % of the ferrous ferric oxide particles. The poly-R-lysine accounts for 6-20 wt. % of the ferrous ferric oxide particles.

### CROSS-REFERENCE TO RELATED APPLICATIONS

This application is a continuation-in-part of International Patent Application No. PCT/CN2015/098234 with an international filing date of Dec. 22, 2015, designating the United States, now pending, and further claims foreign priority benefits to Chinese Patent Application No. 201510870335.3 filed Dec. 1, 2015. The contents of all of the aforementioned applications, including any intervening amendments thereto, are incorporated herein by reference. Inquiries from the public to applicants or assignees concerning this document or the related applications should be directed to: Matthias Scholl P.C., Attn.: Dr. Matthias Scholl Esq., 245 First Street, 18th Floor, Cambridge, Mass. 02142.

### BACKGROUND

The disclosure relates to an MRI contrast agent and a method for preparing the same.

Superparamagnetic iron oxide nanoparticles are a known contrast enhancing agent for magnetic resonance imaging (MRI). However, one problem with conventional iron oxide contrast agent formulations is that they tend to accumulate in the human body.

### SUMMARY

Disclosed is a contrast agent comprising ferrous ferric oxide particles and poly-R-lysine coating.

Disclosed is a contrast agent comprising superparamagnetic nanoparticles and hydroxyethyl starch. The weight ratio of the superparamagnetic nanoparticles to the hydroxyethyl starch is between 1:5 and 1:15; the superparamagnetic nanoparticles have a particle size of 100-140 nm, and comprises, from the inside out, ferrous ferric oxide particles, citric acid, and poly-R-lysine; and the citric acid accounts for 6-13 wt. % of the ferrous ferric oxide particles, and the poly-R-lysine accounts for 6-20 wt. % of the ferrous ferric oxide particles.

The citric acid layer can be coated on the surface of the ferrous ferric oxide particles by means of coordination and adsorption. The poly-R-lysine layer can be ionically bonded to the surface of the citric acid layer. The hydroxyethyl starch can have an average molecule weight of 110-150 kDa.

The contrast agent can be in the form of a lyophilized powder, and can further comprise mannitol that can be 10-30 times the weight of the superparamagnetic nanoparticles.

The contrast agent can be in the form of a solution comprising 10¹ to 10² g/L of the superparamagnetic nanoparticles.

The poly-R-lysine can account for 10-15 wt. % of the ferrous ferric oxide particles.

A method for preparing the contrast agent comprises:

1) preparing a material power comprising 6-12% by weight (wt. %) of ferrous ferric oxide particles having a particle size of 60-75 nm, 0.6-1.2 wt. % of citric acid, and 86.8-93.4 wt. % of hydroxyethyl starch, coating the citric acid on the ferrous ferric oxide particles; formulating the material powder into a 0.04-0.2 wt. % aqueous solution, and removing free iron ions and free citric acid residues in the aqueous solution; and

2) weighing poly-R-lysine accounting for 6-20 wt. % of the ferrous ferric oxide particles, adding the poly-R-lysine to the aqueous solution obtained in 1), and uniformly dispersing the poly-R-lysine in the aqueous solution, the poly-R-lysine being ionically bonded to a surface of the citric acid, to yield the contrast agent.

The material powder is prepared is as follows:

(i) uniformly mixing the ferrous ferric oxide particles having a particle size of 60-75 nm, the citric acid, and N,N-dimethyl formamide at a weight ratio of between 1:0.1:10 and 1:1:100, heating at a temperature of 60-90° C. to dissolve the ferrous ferric oxide particles and allowing the citric acid to coat on surfaces of the ferrous ferric oxide particles; and removing agglomerated ferrous ferric oxide particles, to obtain a solution containing ferrous ferric oxide particles;

(ii) mixing the solution containing ferrous ferric oxide particles obtained in 1), a hydroxyethyl starch solution, and N,N-dimethyl formamide at a weight ratio of between 1:0.1:5 and 1:1:20, stirring and uniformly dispersing a resulting mixture at 60-90° C., to yield a mixed solution comprising 5-20 wt. % of the hydroxyethyl starch solution;

(iii) adding methyl t-butyl ether to the mixed solution obtained in 2), a volume of the methyl t-butyl ether being 2-5 times volume of the mixed solution, and allowing the ferrous ferric oxide particles and the hydroxyethyl starch solution to form a precipitate and

(iv) centrifuging and drying the precipitate obtained in 3), to yield the material powder.

In (1), the free iron ions and free citric acid residues in the aqueous solution can be removed by tangential flow ultrafiltration.

1) can be implemented as follows: transferring the aqueous solution to a storage container of a tangential flow ultrafiltration device, and purifying the aqueous solution by tangential flow ultrafiltration using an ultrafiltration module until a volume ratio between a liquid in a filtrate container to a liquid in the storage container of the tangential flow filtration device is between 2:1 and 2:3.

The method can further comprise: adding mannitol to the contrast agent obtained in (2), a weight of the mannitol being 1 to 2 times weight of the material powder, uniformly mixing the mannitol and the material powder, sterilizing, and lyophilizing, to obtain the contrast agent in the form of a lyophilized powder.

Use of the contrast agent in magnetic resonance imaging (MRI) is also provided.

Advantages of the contrast agent and the method of preparing the same in the disclosure are summarized as below:

1. The contrast agent can reduce the concentration of free iron ions, reduce the adsorption of iron ions on cells and reduce the tissue damage.

2. The ferrous ferric oxide particles coated with the poly-R-lysine layer cannot be absorbed in human, and are rapidly cleared in the body after MRI, leading to a low tissue residue.

3. The MRI contrast agent of the disclosure can reduce the risk of deterioration of liver cirrhosis.

4. The MRI contrast agent can be stored reliably for a long period of time.

### BRIEF DESCRIPTION OF THE DRAWINGS

FIG. 1 is a transmission electron microscopy (TEM) image of Example 2 of the disclosure;

FIGS. 2A-2B show Prussian blue staining analysis of adsorption on cells of iron oxide nanoparticles coated with non-natural poly-R-lysine provided in the disclosure, in which FIG. 2A shows a remarkable positive Prussian blue staining result due to the large adsorption on cells of iron oxide nanoparticles without poly-R-lysine coating; and FIG. 2B shows a very weak positive Prussian blue staining result from the adsorption on cells of iron oxide nanoparticles coated with poly-R-lysine in Example 2;

FIGS. 3A-3D show analysis of magnetic resonance T2 imaging of liver cirrhosis-primary liver tumor in mice with Example 2 of the disclosure; and

FIGS. 4A-4B are Prussian blue staining analysis in subcutaneous tumor tissue of Example 2 of the disclosure.

### DETAILED DESCRIPTION

To further illustrate, experiments detailing a contrast agent and a method for preparing the same are described below. It should be noted that the following examples are intended to describe and not to limit the description.

A contrast agent comprises superparamagnetic nanoparticles and hydroxyethyl starch at a weight ratio of 1:5-1:15. The superparamagnetic nanoparticle has a particle size of 100-140 nm, and comprises, from the inside to the outside, a ferrous ferric oxide particle, a citric acid layer, and a poly-R-lysine layer. The citric acid is adsorbed onto the surface of the ferrous ferric oxide particle, and the poly-R-lysine is ionically bonded to the surface of the citric acid. The citric acid is 6-13% by weight based on the ferrous ferric oxide particle, and the poly-R-lysine is 6-20% by weight based on the ferrous ferric oxide particle. When the poly-R-lysine is 10-15% by weight based on the ferrous ferric oxide particle, the ferrous ferric oxide can be coated more evenly by the poly-R-lysine.

The MRI contrast agent is in the form of a solution or a lyophilized powder. When the contrast agent is a lyophilized powder, mannitol that is 10-30 times the weight of the superparamagnetic nanoparticles may be further added to the contrast agent, such that the MRI contrast agent can be more conveniently stored in the form of a powder at a low temperature. When the contrast agent is a solution, the content of the superparamagnetic nanoparticles is 10⁻³ to 10² g/L. The contrast agent as a solution or a lyophilized powder may serve for different purposes. The lyophilized powder is more convenient for shipping and long-term storage, and the solution is more convenient in use.

A method for preparing the contrast agent is summarized as follows.

(1) A material powder is formulated into a 0.04-0.2 wt. % aqueous solution, and the free iron ions and free citric acid residues contained therein are removed by tangential flow ultrafiltration or dialysis, where the material powder comprises 6-12% by weight (wt. %) of ferrous ferric oxide particles, 0.6-1.2 wt. % of citric acid, and 86.8-93.4 wt. % of hydroxyethyl starch, in which the ferrous ferric oxide particle has a particle size of 60-75 nm, and the citric acid is coated on the surface of the ferrous ferric oxide particle.

(2) A 0.04-0.2% poly-R-lysine solution of 0.01-0.05 time volume of the aqueous solution obtained in (1) is added, where the amount of the poly-R-lysine is 6-20% by weight based on the ferrous ferric oxide particles, such that the poly-R-lysine is fully uniformly dispersed in the solution, and ionically bonded to the surface of the citric acid, to obtain the MRI contrast agent. When less poly-R-lysine is added, the coating on the ferrous ferric oxide particles is not complete, and when more poly-R-lysine is added, the ferrous ferric oxide particle trends to be too large and agglomerate to form a precipitate. The binding reaction time mainly depends on the temperature and the total amount of the reactants. At normal temperature, when the total volume of the solution is about 2500 mL, complete binding can be achieved in a reaction time of 30 min.

(3) The MRI contrast agent is sterilized and then directly stored, to obtain a contrast agent in the form of a solution, or lyophilized and stored, to obtain a contrast agent in the form of a lyophilized powder. When the contrast agent is stored in the form of a lyophilized powder, mannitol of 0.5 to 2 times weight of the material powder is added to the MRI contrast agent, mixed until uniform, sterilized, and then lyophilized, to obtain a contrast agent that can be stored for a longer period of time at a low temperature due to the presence of mannitol. The composition of the MRI contrast agent prepared through the method comprises 2.0-5.0 wt. % of ferrous ferric oxide, 0.2-0.5% of citric acid, 0.2-5.0% of poly-R-lysine, 25-40% of hydroxyethyl starch, and 50-70% of mannitol.

The material powder in (1) is prepared as follows.

(1) Ferrous ferric oxide particles having a particle size of 60-75 nm, citric acid, and N,N-dimethyl formamide are uniformly mixed at a weight ratio of 1:0.1:10-1:1:100, and heated at 60-90° C. until the ferrous ferric oxide particles are completely dissolved, and the citric acid is coated on the surface of the ferrous ferric oxide particles; and the agglomerated ferrous ferric oxide particles are removed, to obtain a solution containing ferrous ferric oxide particles.

(2) The solution containing ferrous ferric oxide particles obtained in (1), a hydroxyethyl starch solution, and N,N-dimethyl formamide are mixed at a weight ratio of 1:0.1:5-1:1:20, and reacted at 60-90° C. with stirring until the materials are completely dispersed uniformly, to obtain a raw material mix solution, where the concentration of the hydroxyethyl starch solution is 5-20 wt. %, and the hydroxyethyl starch has an average molecule weight of 110-150 KDa, and serves to enhance the solubility of the ferrous ferric oxide particles.

(3) Methyl t-butyl ether of 2-5 times volume of the raw material mix solution is added to the raw material mix solution obtained in (2), such that the ferrous ferric oxide particles in the raw material mix solution form a precipitate with the hydroxyethyl starch.

(4) The precipitate obtained in (3) is centrifuged and dried, to obtain the material powder.

In (1), the tangential flow ultrafiltration is specifically as follows. The aqueous solution is transferred to a storage container of a tangential flow ultrafiltration device, and purified by tangential flow filtration using an ultrafiltration module until the ratio between the volume of liquid in a filtrate container to the volume of liquid in the storage container of the tangential flow filtration device is between 2:1-2:3, upon which the liquid in the storage container of the filtration device is collected. A larger volume of liquid in the filtrate container indicates a greater number of cycles of tangential flow filtration and purification, and a greater extent of removal of the iron ions and free citric acid residues. However, when excessive liquid exists in the filtrate container, the active ingredients in the material powder may be caused to lose easily. The ratio between the volume of the liquid in the filtrate container to the volume of liquid in the storage container is set between 2:1-2:3, such that the free iron ions and free citric acid residues are completely removed, without causing too much loss of the material powder.

The MRI contrast agent is recommended to be used at a dosage of 0.2-1 mg/kg of body weight based on iron.

### Example 1: Method for Preparing Ferrous Ferric Oxide Nanoparticles Coated with Poly-R-Lysine

(1) 5 g of a solid material powder (comprising 9.2 wt. % of superparamagnetic ferrous ferric oxide nanoparticles, 0.8 wt. % of citric acid, and 88.5% of hydroxyethyl starch having an average molecule weight of 130 KDa, with the balance being the impurities in the material powder) was weighed, dissolved in 5.0 L of pure water, then transferred to a storage container of a tangential flow ultrafiltration device (Millipore Pellicon 2), and purified by tangential flow ultrafiltration using an ultrafiltration module (a fiber membrane with a molecule weight cutoff of 5 k fitted in Millipore Pellicon 2). In the process, the tangential flow velocity was set to a flow velocity at a critical point between a linear pressure difference and a saturated pressure difference of the tangential flow (10 mL/min). When the volume of liquid in the filtrate container of the tangential flow filtration device was 2500 mL, the liquid in the storage container of the filtration device was collected.

(2) 1 g of solid poly-R-lysine was weighed, and 999 g of ultrapure water was added to prepare a 0.1% poly-R-lysine solution. The liquid obtained in (1) was stirred and 50 mL of the poly-R-lysine solution was gradually added and stirred for 30 min.

(3) 4.2 g of mannitol was added.

(4) The liquid obtained in the above step was filtered through a 0.22 μm-pore size filter membrane and the resulting solution was frozen to form a solid, which was then lyophilized under vacuum. The resulting powder was nanoscale superparamagnetic solid ferrous ferric oxide coated with poly-R-lysine.

In (1), the material powder was prepared following the method for preparing nanoscale superparamagnetic solid ferrous ferric oxide containing hydroxyethyl starch as described in Chinese Patent No. ZL2013 1 0284215.6 entitled “Solution comprising stable nanocale superparamagnetic ferrous ferric oxide and preparation method and use thereof”.

### Example 2: Lyophilized Powder Injection of Nanoscale Ferrous Ferric Oxide Coated with Poly-R-Lysine

The liquid obtained in (2) of Example 1 was filtered through a 0.22 μm-pore size filter membrane, packaged in a glass vial having a volume of 5 mL, and then lyophilized under vacuum. The powder obtained in the glass vial was sealed by capping under nitrogen, to obtain a lyophilized powder injection of nanoscale ferrous oxide coated with poly-R-lysine. When the powder injection was used, 3.5 mL of physiological saline was injected into the glass vial and shaken to prepare a solution. The recommended dose was 0.5 mL per 10 kg of body weight.

### Example 3

(1) The operations in Example 1 were repeated, except that in (1), when the volume of liquid in the filtrate container of the tangential flow filtration device was 1700 mL, the liquid in the storage container of the filtration device was collected.

(2) 67 mL of 0.04% poly-R-lysine solution was added and stirred for 30 min.

(3) The liquid was sterilized by filtration and then directly packaged and sealed in a 20 mL glass vial for storage.

### Example 4

(1) The operations in Example 1 were repeated, except that in (1), when the volume of liquid in the filtrate container of the tangential flow filtration device was 3000 mL, the liquid in the storage container of the filtration device was collected.

(2) 45 mL of 0.2% poly-R-lysine solution was added and stirred for 30 min.

(3) 2.5 g of mannitol was added, sterilized by filtration, and then packaged and sealed in a 5 mL glass vial for storage.

### Example 5

(1) The operations in Example 1 were repeated, except that in (1), the iron ions and free citric acid residues in the solution were removed by dialysis.

(2) 45 mL of 0.2% poly-R-lysine solution was added and stirred for 30 min.

(3) 10 g of mannitol was added, sterilized by filtration, and then packaged and sealed in a 5 mL glass vial for storage.

### Comparison Example

5 g of a solid material powder (comprising 9.2 wt. % of superparamagnetic ferrous ferric oxide nanoparticles, 0.8 wt. % of citric acid, and 88.5% of hydroxyethyl starch, with the balance being the impurities in the material powder) was weighed, dissolved in 5.0 L of pure water, to obtain a material powder solution. 1 g of solid poly-L-lysine was weighed, and 999 g of ultrapure water was added to prepare a 0.1% poly-L-lysine solution. The material powder solution was stirred and 50 mL of the poly-L-lysine solution was gradually added and stirred for 30 min.

Analysis of Experimental Results

Main chemicals and reagents: available from Sigma-Aldrich, J & K Scientific, Aladdin Reagents, and Sinopharm Reagents, etc.

Main instruments and equipment: freeze-drier (Labconco), Nano-ZS90 dynamic laser scattering apparatus (Malvern), transmission electron microscope (H-7000FA, Hitachi, Japan), magnetic resonance imaging machine (Siemens Magnetom Trio Tim 3.0T), and SpectrAA-40 atomic absorption spectrometer (VARIAN, USA).

Analysis of free iron ions in samples provided in Example 1

5.0 mL of the solution obtained in (1) of Example 1 was transferred to the Amicon Ultra-15 ultrafiltration device from Millpore, and centrifuged at 4000 g for 10 minutes. The ferric ions present in the collected filtrate were reduced into ferrous ions by using hydroxylamine hydrochloride as a reducing agent, which were then reacted with o-phenanthroline as a chromogenic reagent at about pH 5. Then, the absorption at a wavelength of 530 nm was determined. The result shows that the total concentration of ferrous and ferric iron ions in the solution obtained by tangential flow filtration in (1) of Example 1 was lower than the minimum detection limit of the ferrous ion-standard curve method (<50 ppm by weight). In contrast, the iron ion content in the solution not amenable to tangential flow filtration is 1.15% based on the total weight. This result indicates that the free iron ions have been effectively removed by tangential flow filtration.

Particle size analysis of nanoparticles in solution formulated with nanoscale ferrous ferric oxide lyophilized powder coated with poly-R-lysine provided in Example 2

150 mg of the lyophilized solid powder obtained in Example 2 was accurately weighed and then 5.0 mL of physiological saline was added. After the solid was completely dissolved, the solution was 10-fold diluted with pure water. The particle size, distribution, and zeta potential were measured with 2.0 mL of the diluted solution by using Nano-ZS90 dynamic laser scattering apparatus at 25° C. The results are shown in Table 1.

TABLE 1

| | Average particle size | Polydispersity Index |
|---|---|---|
| Sample 1 | 126.3 nm | 0.114 |
| Sample 2 | 124.9 nm | 0.161 |
| Sample 3 | 127.4 nm | 0.159 |
| Sample 4 | 126.5 nm | 0.141 |
| Sample 5 | 122.4 nm | 0.126 |

The results of particle size analysis show that the particle size distribution of the nanoparticles coated with poly-R-lysine is normal distribution with an average particle size of 125.5 nm.

TEM analysis of Nanoparticles in solution formulated with nanoscale ferrous ferric oxide lyophilized powder coated with poly-R-lysine provided in Example 2

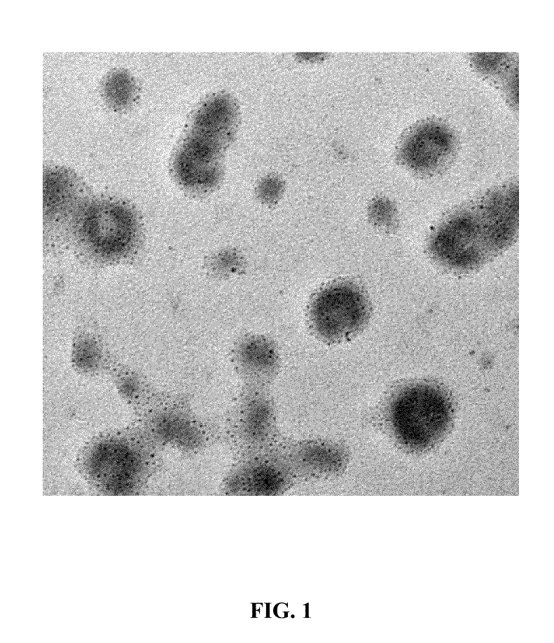

150 mg of the lyophilized solid powder obtained in Example 2 was accurately weighed and then 5.0 mL of physiological saline was added. After the solid was completely dissolved, a portion of the solution was prepared into a TEM sample, and then observed under a transmission electron microscope (H-7000FA, Hitachi, Japan). The sample has good dispersivity and the TEM image is shown in FIG. 1. The TEM analysis shows that the nanoparticles are distributed in a nanocluster format, and the size is consistent with the results from the dynamic laser scattering apparatus. Therefore, the disclosure provides novel ferrous ferric oxide nanoparticles coated with poly-R-lysine.

Analysis of iron content in nanoscale ferrous ferric oxide lyophilized powder coated with poly-R-lysine provided in Example 2

150 mg of the lyophilized solid powder sample obtained in Example 2 was accurately weighed and then 5.0 mL of physiological saline was added. After the solid was completely dissolved, the solution was 1000-fold diluted with pure water. The iron content in 25.0 mL of the diluted solution was determined by the SpectrAA—40 atomic absorption spectrometer. The result is shown in Table 2.

TABLE 2

| | Sample 1 | Sample 2 | Sample 3 | Sample 4 | Sample 5 | Average |
|---|---|---|---|---|---|---|
| Iron in the diluent (mg/L) | 0.729 | 0.740 | 0.732 | 0.742 | 0.711 | 0.731 |
| Iron content (mg) | 3.65 | 3.70 | 3.66 | 3.71 | 3.56 | 3.66 |

The result of measurement by atomic absorption spectrometry indicates that the average iron content in each sample is 2.44% by weight.

Based on the above measurement results and the ratio of the raw materials in the preparation process, it can be calculated that the superparamagnetic nanoscale ferrous ferric oxide solid coated with poly-R-lysine prepared in Example 1 comprises 3.3 wt. % of ferrous ferric oxide, 0.3% of citric acid, 0.5% non-natural poly-R-lysine, 31.8% of hydroxyethyl starch, and 63.1% of mannitol. After multiple repeated experiments following the methods in Examples 1-5, the content of each component in the MRI contrast agents obtained is: ferrous ferric oxide 2.0-5.0 wt. %, citric acid 0.2-0.5 wt. %, poly-R-lysine 0.2-5.0 wt. %, hydroxyethyl starch 25-40 wt. %, and mannitol 50-70 wt. %.

Adsorption on cells of ferrous ferric oxide nanoparticles in a solution prepared with the lyophilized powder provided in Example 2

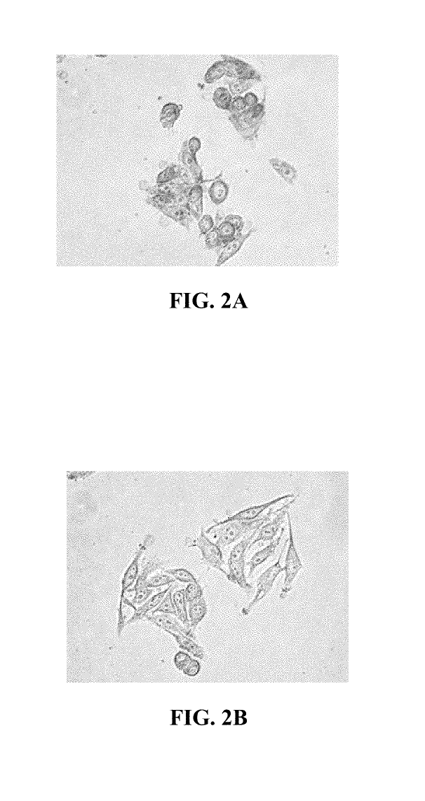

150 mg of the lyophilized solid powder obtained in Example 2 was accurately weighed and then 5.0 mL of physiological saline was added. After the solid was completely dissolved, 100 μl of the solution was added to a culture medium containing the adherent cell line BEL-7402. After one hour of incubation, the cell culture medium was aspirated. The adherent cells were gently washed twice with physiological saline and fixed with paraformaldehyde. The ferrous ferric oxide particles adsorbed on the cells were stained with Prussian blue and then analyzed by microscopy. The adsorption on cells of nanoscale ferrous ferric oxide without poly-R-lysine coating was used as a reference control. The results are shown in FIG. 2A-2B. The results show that the adsorption on cells of the nanoscale ferrous ferric oxide without poly-R-lysine coating gives rise to a remarkable positive Prussian blue staining result (FIG. 2A), and a very weak positive Prussian blue staining result is displayed on cells with the nanoscale ferrous ferric oxide coated with poly-R-lysine (FIG. 2B). Therefore, by using the method for coating nanoscale ferrous ferric oxide with poly-R-lysine provided in the disclosure, the non-specific adsorption on cells can be effectively reduced.

Determination and analysis of iron contents in various organs in mice injected with an injectable solution formulated with nanoscale ferrous ferric oxide lyophilized powder coated with poly-R-lysine provided in Example 2

150 mg of the lyophilized solid powder obtained in Example 2 was accurately weighed and then 5.0 mL of physiological saline was added. After the solid was completely dissolved, according to the body weight of the mice (three mice per group), the prepared solution was intravenously injected at a dose of 2.0 mg/kg body weight based on iron. The iron contents in serum, liver and spleen were determined and analyzed at 0.5, 3, and 24 hours after the injection, and the mass distribution of iron in each sample is calculated. The results are shown in Table 3.

TABLE 3

| | Injected sample and duration | | | | | | |
|---|---|---|---|---|---|---|---|
| | Example 2 | | | Comparison Example | | | Control |
| | 0.5 h | 3 h | 24 h | 0.5 h | 3 h | 24 h | group |
| Serum iron | 7.5 ± 1.0 | 6.6 ± 0.3 | 3.5 ± 1.0 | 8.4 ± 0.9 | 8.9 ± 1.7 | 3.8 ± 0.2 | 3.0 ± 0.2 |
| (μg/mL) | | | | | | | |
| Liver iron | 150.6 ± 5.6 | 140.3 ± 30.9 | 91.7 ± 11.5 | 133.5 ± 18.4 | 155.4 ± 13.6 | 94.6 ± 26.0 | 86.2 ± 6.6 |
| (μg/mL) | | | | | | | |
| Spleen iron | 171.8 ± 9.9 | 153.5 ± 11.1 | 153.0 ± 12.2 | 187.1 ± 23.8 | 220.0 ± 23.0 | 158.7 ± 28.7 | 150.7 ± 20.2 |
| (μg/mL) | | | | | | | |

The results show that the nanoscale ferrous ferric oxide coated with poly-R-lysine is cleared rapidly in the spleen, and returned to normal 3 hours after injection. In contrast, the nanoscale ferrous ferric oxide coated with natural poly-L-lysine is still significantly higher than normal in the spleen 3 hours after injection. In the liver, the content of the nanoscale ferrous ferric oxide coated with poly-R-lysine is also lower than that of the nanoscale ferrous ferric oxide coated with natural poly-L-lysine at the time point of 3 hours after injection. Therefore, the nanoscale ferrous ferric oxide coated with poly-R-lysine has a unique characteristic of rapid clearance in visceral organs.

Serological determination and analysis of liver and kidney function indices in liver cirrhosis model in mice injected with an injectable solution formulated with nanoscale ferrous ferric oxide lyophilized powder coated with poly-R-lysine provided in Example 2

The liver cirrhosis model in mice was established according to the method reported by Chang M L et al. (World Journal of Gastroenterology 2005, 11, 4167). After the liver cirrhosis model in mice was established, 150 mg of the lyophilized solid powder obtained in Example 2 was accurately weighed and then 5.0 mL of physiological saline was added. After the solid was completely dissolved, according to the body weight of the mice, the prepared solution was intravenously injected at a dose of 2.0 mg/kg body weight based on iron. Then, the liver and kidney function indices were serologically determined before and 1, 3, and 5 days after injection. The liver and kidney function indices of normal mice were used as a reference control. The results are shown in Table 4.

TABLE 4

| | | | Cirrhotic mice | Cirrhotic mice | Cirrhotic mice |
|---|---|---|---|---|---|
| | Normal | Cirrhotic mice | First day after | Third day after | Fifth day after |
| Serum indexes | mice | Before injection | injection | injection | injection |
| Alanine transaminase | 52.0 | 177.50 | 212.5 | 110.0 | 57.50 |
| Aspartate aminotransferase | 84.0 | 131.3 | 144.3 | 114.3 | 85.5 |
| Alkaline phosphatase | 122.8 | 142.0 | 156.7 | 154.8 | 138.5 |
| Total bilirubin | 1.04 | 2.33 | 1.87 | 1.84 | 1.10 |
| Glucose | 6.43 | 10.28 | 9.60 | 10.70 | 9.11 |
| Blood urea nitrogen | 6.63 | 7.38 | 7.23 | 6.46 | 6.33 |
| Creatinine | 17.98 | 11.8 | 15.7 | 13.7 | 15.6 |

The test result shows that after an injectable solution formulated with nanoscale ferrous ferric oxide lyophilized powder coated with poly-R-lysine provided in Example 2 is injected, the liver function indices of liver cirrhosis model mice is slightly elevated after 24 hrs, and the liver and kidney function indices are consistent with that of normal mice 5 days after injection. The result indicates that the injection formulated with nanoscale ferrous ferric oxide lyophilized powder coated with poly-R-lysine will not cause further damage in the liver cirrhosis model.

Analysis of magnetic resonance imaging in liver cirrhosis-primary liver tumor model with an injectable solution formulated with nanoscale ferrous ferric oxide lyophilized powder coated with poly-R-lysine provided in Example 2

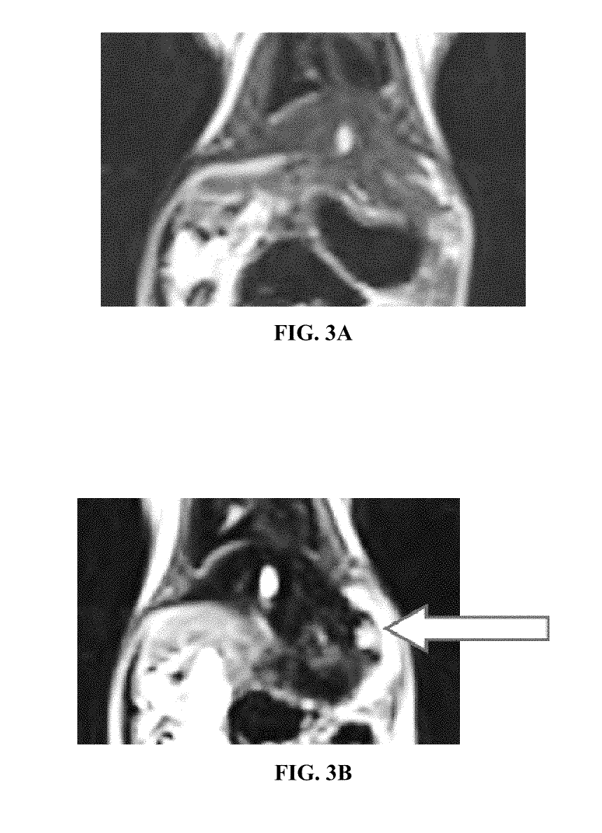

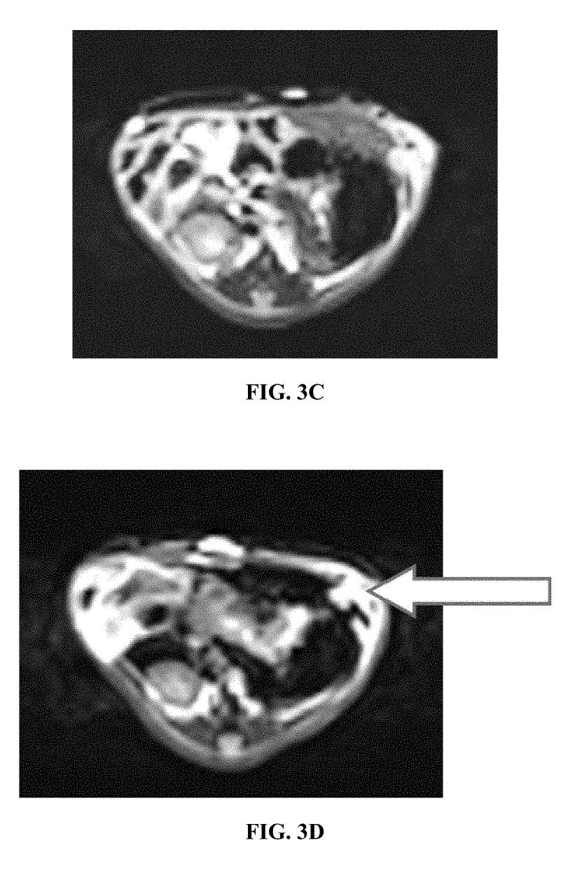

The animal model of liver cirrhosis-primary liver tumor in mice was established by surgically transplanting liver tumor tissue to cirrhotic liver in mice. After the model was established, 150 mg of the lyophilized solid powder obtained in Example 2 was accurately weighed and then 5.0 mL of physiological saline was added. After the solid was completely dissolved, according to the body weight of the mice, the prepared solution was intravenously injected at a dose of 0.5 mg/kg body weight based on iron. Then T2 imaging scan and image analysis were performed on an NMR instrument. The MRI scan was conducted in two batches of experiments before and after injection. The results are shown in FIGS. 3A-3D.

Before the contrast agent is injected, as shown in FIGS. 3A and 3B, the tumor is obscure in MRI and it is difficult to determine the presence and size of the tumor. After the injectable solution formulated with nanoscale ferrous ferric oxide lyophilized powder coated with poly-R-lysine provided in Example 2 is injected, as shown in FIGS. 3C and 3D, the T2 image of the tumor in MRI shows the location, boundary and size of the tumor (as shown by the arrow in FIGS. 3B and 3D). Therefore, after the injectable solution formulated with nanoscale ferrous ferric oxide lyophilized powder coated with poly-R-lysine provided in Example 2 is injected, the primary liver tumor in liver cirrhosis is facilitated to be positioned, and the imaging effect is significant.

Analysis of distribution in other tumor models of an injectable solution formulated with nanoscale ferrous ferric oxide lyophilized powder coated with poly-R-lysine provided in Example 2

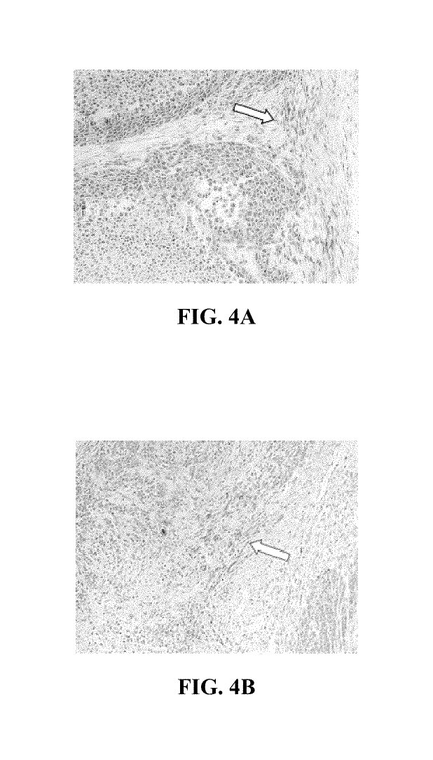

The subcutaneous tumor models were established by transplanting tumor cells subcutaneously in mice, and mainly included subcutaneous pancreatic and liver tumors. After the model was established, 150 mg of the lyophilized solid powder obtained in Example 2 was accurately weighed and then 5.0 mL of physiological saline was added. After the solid was completely dissolved, according to the body weight of the mice, the prepared solution was intravenously injected at a dose of 0.5 mg/kg body weight based on iron. At 24 hrs after injection, the tumor tissues were collected, sliced and stained with Prussian blue. Microscopic images were taken for analysis. The results are shown in FIG. 4A-4B. FIG. 4A shows the subcutaneous pancreatic cancer tumor tissue, and FIG. 4B shows the subcutaneous liver tumor tissue, where the arrows indicate a positive Prussian blue staining result. The result shows that the nanoscale ferrous ferric oxide coated with poly-R-lysine provided in the disclosure has a certain distribution at the interface of subcutaneous tumor tissues.

Unless otherwise indicated, the numerical ranges involved include the beginning and end values. It will be obvious to those skilled in the art that changes and modifications may be made, and therefore, the aim in the appended claims is to cover all such changes and modifications.

What is claimed is:

1. A contrast agent, comprising:
1) superparamagnetic nanoparticles; and
2) hydroxyethyl starch;
wherein:
a weight ratio of the superparamagnetic nanoparticles to the hydroxyethyl starch is between 1:5 and 1:15;
the superparamagnetic nanoparticles have a particle size of 100-140 nm, and comprise, from the inside out of the superparamagnetic nanoparticles: ferrous ferric oxide particles, citric acid, and poly-R-lysine; and
the citric acid accounts for 6-13 wt. % of the ferrous ferric oxide particles, and the poly-R-lysine accounts for 6-20 wt. % of the ferrous ferric oxide particles.

2. The contrast agent of claim 1, being in the form of a lyophilized powder, and further comprising mannitol that is 10-30 times the weight of the superparamagnetic nanoparticles.

3. The contrast agent of claim 1, being in the form of a solution comprising 10⁻³ to 10² g/L of the superparamagnetic nanoparticles.

4. The contrast agent of claim 1, wherein the poly-R-lysine accounts for 10-15 wt. % of the ferrous ferric oxide particles.

5. A method for preparing the contrast agent of claim 1, the method comprising:
1) preparing a material power comprising 6-12% by weight (wt. %) of ferrous ferric oxide particles having a particle size of 60-75 nm, 0.6-1.2 wt. % of citric acid, and 86.8-93.4 wt. % of hydroxyethyl starch, coating the citric acid on the ferrous ferric oxide particles; formulating the material powder into a 0.04-0.2 wt. % aqueous solution, and removing free iron ions and free citric acid residues in the aqueous solution;
2) weighing poly-R-lysine accounting for 6-20 wt. % of the ferrous ferric oxide particles, adding the poly-R-lysine to the aqueous solution obtained in 1), and uniformly dispersing the poly-R-lysine in the aqueous solution, the poly-R-lysine being ionically bonded to a surface of the citric acid, to yield the contrast agent.

6. The method of claim 5, wherein the material power is prepared as follows:
i) uniformly mixing the ferrous ferric oxide particles having a particle size of 60-75 nm, the citric acid, and N,N-dimethyl formamide at a weight ratio of between 1:0.1:10 and 1:1:100, heating at a temperature of 60-90° C. to dissolve the ferrous ferric oxide particles and allowing the citric acid to coat on surfaces of the ferrous ferric oxide particles; and removing agglomerated ferrous ferric oxide particles, to obtain a solution containing ferrous ferric oxide particles;
ii) mixing the solution containing ferrous ferric oxide particles obtained in 1), a hydroxyethyl starch solution, and N,N-dimethyl formamide at a weight ratio of between 1:0.1:5 and 1:1:20, stirring and uniformly dispersing a resulting mixture at 60-90° C., to yield a mixed solution comprising 5-20 wt. % of the hydroxyethyl starch solution;
iii) adding methyl t-butyl ether to the mixed solution obtained in 2), a volume of the methyl t-butyl ether being 2-5 times volume of the mixed solution, and allowing the ferrous ferric oxide particles and the hydroxyethyl starch solution to form a precipitate; and
iv) centrifuging and drying the precipitate obtained in 3), to yield the material powder.

7. The method of claim 5, wherein in 1), the free iron ions and free citric acid residues in the aqueous solution are removed by tangential flow ultrafiltration.

8. The method of claim 7, wherein 1) is implemented as follows: transferring the aqueous solution to a storage container of a tangential flow ultrafiltration device, and purifying the aqueous solution by tangential flow ultrafiltration using an ultrafiltration module until a volume ratio between a liquid in a filtrate container to a liquid in the storage container of the tangential flow filtration device is between 2:1 and 2:3.

9. The method of claim 5, further comprising: adding mannitol to the contrast agent obtained in (2), a weight of the mannitol being 1 to 2 times weight of the material powder, uniformly mixing the mannitol and the material powder, sterilizing, and lyophilizing, to obtain the contrast agent in the form of a lyophilized powder.

10. The method of claim 5, wherein the contrast agent is in the form of a lyophilized powder, and further comprising mannitol that is 10-30 times the weight of the superparamagnetic nanoparticles.

11. The method of claim 5, wherein the contrast agent is in the form of a solution comprising 10⁻³ to 10² g/L of the superparamagnetic nanoparticles.

12. The method of claim 5, wherein the poly-R-lysine accounts for 10-15 wt. % of the ferrous ferric oxide particles.
